(19) United States
(12) Patent Application Publication
(10) Pub. No.: US 2004/0033936 A1
(43) Pub. Date: Feb. 19, 2004

(54) Production, stabilisation and use of reduced forms of pharmaceutical compounds

(76) Inventors: Ernst Wulfert, Brussels (BE); Anthony Atkinson, Hampshire (GB); Andres Marc Salomon, Wiltshire (GB)

Correspondence Address: Baker & Hostetler, 1050 Connecticut Avenue N W, Washington Square, Suite 1100, Washington, DC, 20036, US

(21) Appl. No.: 10/311,152

(22) PCT Filed: Jul. 10, 2001
(86) PCT No.: PCT/GB01/03102

(30) Foreign Application Priority Data
Jul. 11, 2000 (GB) 0017060.5

(51) Int. Cl. A61K 38/06; A61K 38/05; A61K 31/5415; A61K 31/375; A61K 31/198

(52) U.S. Cl. 514/2; 514/18; 514/224.8; 514/474; 514/562

(57) ABSTRACT

The reduced or “leuco” forms of certain pharmaceutical compounds can be produced and/or stabilised by mixing the unreduced compounds or the reduced compounds, respectively, with acid and a sulphydryl compound, especially a sulphur-containing amino acid, or peptide or derivative of such an amino acid.

The present invention relates to the production, stabilisation and use of reduced forms of pharmaceutical compounds, more particularly to the stabilisation of the reduced (“leuco”) forms of those compounds, and especially to the production, stabilisation and use of reduced forms of the thiazines, and more particularly the phenothiazines.

A number of compounds of pharmaceutical interest can exist in both oxidised and reduced, or leuco, forms, and can be converted with relative ease from one form to the other. Several such compounds were originally used as dyes, and dyes having this characteristic were called ‘vat dyes’. Certain of these vat dyes and a number of closely related compounds, generally characterised by a coloured oxidised form and colourless (or nearly colourless) leuco form have been found to have pharmaceutical activity. It is often desirable to be able to administer such a pharmaceutical compound in the reduced form. This may be because the reduced form is more active than the oxidised form, or because the reduced form is the only biologically active species of the compound. Alternatively, or in addition, the oxidised form of the compound may be more toxic to the patient than the reduced form, and so direct administration of the reduced form may ameliorate one or more side effects associated with the compound.

Even when an oxidised form of the compound is converted to a reduced form within the body of the patient, it may still be desirable to administer the reduced form directly. The direct administration of the reduced form may enable the compound to achieve its therapeutic activity more quickly, and/or to reduce a subject's exposure to a (more) toxic oxidised form. It may also enable the treatment of individuals who have a deficiency in the reductive pathway leading from the administered oxidised form to the active leuco form.

Examples of pharmaceutical compounds which have an oxidised and a reduced form include the phenothiazines, e.g. Toluidine Blue O (tolonium chloride), Thionine, Azure A, Azure B, Azure C, Methylene Blue and 1,9-Dimethyl-methylene Blue. All of these compounds have in common the phenothiazine skeleton, and have a stable, but inactive, oxidised form and an active, but unstable, leuco form.

Other pharmaceutically active compounds which may be stabilised by the process of the present invention include riboflavin, the ubiquinones, 4,7-phenanthroline-5, 6-hydroquinone and dapsone.

The present inventors have discovered a novel method for the conversion of a pharmaceutical compound from an oxidised form to a reduced form and/or for the stabilisation of that compound in a reduced state.

Thus, in a first aspect, the present invention provides a method of reducing an oxidised form of a pharmaceutical compound, by admixing the oxidised form of the compound with ascorbic acid and with at least one sulphydryl compound.

The invention further provides a method of stabilising the reduced form of a pharmaceutical compound which can exist in both oxidised and reduced forms by mixing said pharmaceutical compound with ascorbic acid and with at least one sulphydryl compound.

By “pharmaceutical compound” we mean any compound intended for administration to the human or animal body in a method of medical treatment, which treatment may include prophylaxis. The pharmaceutical compound should be able to exist in at least two oxidation states, and may be able to exist in more than two oxidation states. Where it can exist in more than two oxidation states, the method of reduction involves a conversion of the pharmaceutical compound from a higher oxidation state to a lower (more reduced) oxidation state.

The pharmaceutical compound may be reduced at one or more inorganic and/or organic centres, each of which may exist in two or more states of oxidation. By way of example, reference may be made to metal ions, and to groups containing oxygen, nitrogen and sulphur atoms.

The oxidised form of the compound which is reduced in the method according to the invention may or may not, in itself, possess pharmaceutical or therapeutic activity. If it does have pharmaceutical or therapeutic activity, such activity may be less than that of the reduced form of the compound into which it is converted. Alternatively, or in addition, the oxidised form may or may not show more toxicity to a patient than the reduced form into which it is converted.

The term “pharmaceutical compound” is also intended to encompass prodrugs which have two or more oxidation states. Prodrugs are compounds which have little or no biological activity (whether in an oxidised or a reduced state) but which can be converted into a therapeutic form (the active drug) by a mechanism other than a simple loss or gain of electron(s) in an oxidation-reduction reaction. By way of example, a prodrug may be converted into an active drug by the cleavage of a bond within it, e.g. by the cleavage of a bond by an enzyme, e.g. the cleavage of a peptide bond by a peptidase. The method of the invention may be used to convert a prodrug from a more oxidised to a less oxidised (reduced) state. In the reduced state, the prodrug may be more susceptible to conversion into the active drug than when in the oxidised state.

The sulphydryl compound used in the present invention may be any compound having an —SR group, wherein S represents sulphur and R represents a hydrogen atom or a lower alkyl group, preferably having from 1 to 4 carbon atoms. The —SH group is sometimes referred to as a ‘mercapto group’ and the two terms, ‘mercapto’ and ‘sulphydryl’, are sometimes used interchangeably. The stabilisation of the present invention results in oxidation of the sulphydryl compound of the stabiliser, and it is preferred that the sulphydryl compound is such that the —SH or —SR group is oxidised to a group of formula —S—S —. Preferred sulphydryl compounds are sulphur-containing amino acids and peptides, preferably oligopeptides, including at least one amino acid unit derived from such an amino acid, as well as derivatives of such amino acids and peptides, including salts, esters and amides thereof.

Preferred such amino acids include cysteine, methionine and ethionine. An example of a peptide including a unit derived from such an amino acid is glutathione. An example of a derivative (amide) of such an amino acid is N-acetylcysteine. Thus, preferred sulphydryl compounds are glutathione, cysteine, N-acetyl cysteine, methionine, ethionine, and mixtures of any two or more thereof.

The sulphydryl compound may be admixed with the pharmaceutical compound before, after or simultaneously with the mixing of the pharmaceutical compound with the ascorbic acid. The pharmaceutical compound may alternatively be admixed with a composition containing ascorbic acid and at least one sulphydryl compound.

In the method of reducing a pharmaceutical compound according to the first aspect of the invention, the ascorbic acid may be admixed with the pharmaceutical compound in a weight ratio of from about 10:1 to about 100:1. The sulphydryl compound(s) may be mixed with the pharmaceutical compound in a weight ratio of from about 2:1 to about 200:1. The weight ratio of the sulphydryl compound to ascorbic acid may be from about 1:0.5 to about 1:5.

The method of reduction according to the first aspect of the invention may result in the conversion of some or all of a pharmaceutical compound into a more reduced oxidation state. By way of example, more than 10 percent, more than 20 percent, more than 30 percent, more than 40 percent, more than 50 percent, more than 60 percent, more than 70 percent, more than 80 percent, more than 90 percent, or more than 95 percent of the pharmaceutical compound may be converted into a more reduced form.

The oxidised form of the pharmaceutical compound which is reduced in accordance with the invention may be present within a mixture or composition. The mixture or composition may comprise any of the known types of substance which are traditionally used in pharmaceutical compositions and medicaments. Further substances may be admixed with the composition after the pharmaceutical compound has been reduced. Examples of substances which may be added to the oxidised and/or reduced form of the pharmaceutical compound are described elsewhere herein.

In various further aspects, the present invention provides a pharmaceutical compound which has been reduced by a method according to the invention, a composition containing such a compound, and a method of manufacturing a pharmaceutical composition or medicament comprising admixing such a compound or a composition with one or more pharmaceutically acceptable excipients, carriers, buffers, diluents, or preservatives. The pharmaceutical compound, composition or medicament may be used in a method of medical treatment.

A composition or medicament according to, produced by, or for use in the present invention preferably contains ascorbic acid and at least one sulphydryl compound. The sulplydryl compound may be selected from the group consisting of glutathione, cysteine, N-acetylcysteine, methionine, ethionine, and mixtures thereof. The amount of ascorbic acid relative to the amount of the pnarmaceuticai compound may be from about 10:1 to about 100:1 by weight. The amount of sulphydryl compound(s) may be from about 2:1 to about 200:1 by weight. The weight ratio of the sulphydryl compound to ascorbic acid may be from about 1:0.5 to about 1:5.

The pharmaceutically acceptable excipients, carriers, buffers, diluents and preservatives that may be mixed with the (oxidised or reduced forms of the) pharmaceutical compound or composition containing it should ideally be non-toxic and should preferably not interfere with the activity of the pharmaceutical compound. The precise nature of any excipient, carrier, buffer, diluent, preservative or other material within a composition or medicament may depend on the intended route of administration. Such materials are, however, well known to those skilled in the art and require no further explanation here.

A pharmaceutical composition or medicament of the invention that is ready for storage or administration may be in any suitable form, e.g. in the form of a tablet, capsule, powder, solution, suspension, or emulsion.

Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be used, alone or in combination with other carriers.

The pharmaceutical composition may be in the form of a parenterally acceptable aqueous solution which is pyrogen-free and has suitable pH and isotonicity. Those skilled in the art are well able to prepare suitable solutions using, for example, isotonic vehicles such as sodium chloride, Ringer's injection, or lactated Ringer's Injection.

Where the composition is in the form of a liquid, e.g. a solution, it may be degassed or sparged with an inert gas such as nitrogen or a noble gas (e.g. argon). Degassing or sparging may improve the stability of the reduced form of the pharmaceutical compound to re-oxidation. A liquid composition may be stored under an inert gas such as nitrogen or argon. It may be contained within an airtight biodegradable capsule which is suitable for administration.

Where the composition is a tablet, the pharmaceutical compound may be reduced in solution. The tablet may be obtained by e.g. spray drying techniques which are well known to those skilled in the art. Such spray drying may occur under nitrogen or another inert gas in order to assist in maintaining the pharmaceutical compound in the reduced form. Tablets may be stored in airtight capsules, containers or packs (e.g. blister packs) to decrease their exposure to atmospheric oxygen. Such capsules, containers and packs are well known to those of skill in the art.

In still further aspects, the invention extends to the use of a pharmaceutical compound which has been reduced in accordance with a method of the present invention, or of a pharmaceutical composition containing such a compound, in a method of medical treatment, to the use of such a compound or composition for the manufacture of a medicament for use in such a treatment, and to a method comprising administering such a compound or composition to a subject, e.g. for treatment (which may include preventative treatment) of a disease.

The subject may be an animal, particularly a mammal, which may be human or non-human, such as rabbit, guinea pig, rat, mouse or other rodent, cat, dog, pig, sheep, goat, cattle or horse, or which is a bird, such as a chicken.

Administration of the pharmaceutical compound or composition is preferably in a “prophylactically effective amount” or a “therapeutically effective amount” as the case may be (although prophylaxis may be considered therapy) such an amount being sufficient to show benefit to the subject. The actual amount administered, and rate and time-course of administration, will depend on the nature and severity of what is being treated. Prescription of treatment, e.g. decisions on dosage etc, is within the responsibility of e.g. general practitioners and other medical doctors.

As an important part of the present disclosure, the pharmaceutical compound which is employed in any one of the aspects of the present invention may be a phenothiazine. The phenothiazine may be selected from the group consisting of Toluidine Blue 0, Thionine, Azure A, Azure B, Azure C, Methylene Blue and 1,9-Dimentyl-methylene Blue, and mixtures thereof.

The present invention is not however restricted to such compounds and may relate to other classes of compounds, e.g. to drugs described elsewhere herein which have an oxidised and a reduced state. Examples include ubi and dapsone.

The reduced forms of these compounds may be used to treat a variety of disorders, including those for which the oxidised forms are known treatment and others.

Thus, the reduced forms of these compounds may be used in the prophylaxis and treatment of methaemoglobinaemia, for which methylene blue, and sometimes riboflavin, is the drug of choice, based on the discovery that it is the reduced, leuco, form of these compounds which is the active species. The compounds may also be used in the prophylaxis and treatment of disorders resulting from oxidative injury, such as Parkinson's Disease, myocardial infarction and stroke. The use of the compounds of the present invention in the prophylaxis and treatment of these disorders forms the subject of an application filed on the same date as this application.

Other disorders which may be treated, prevented or alleviated by the compounds of the present invention include Alzheimer's Disease, motor neurone disease, Lewy Body disease, Pick's disease and Progressive Supranuclear Palsy.

Without wishing to be bound by theory, it is believed that it is the leuco forms of the phenothiazines which are able to cross the blood-brain barrier. It is also believed that it is the oxidised (non-leuco) form of the phenothiazines that is responsible for the mutagenicity and toxicological problems that are observed in the literature.

Aspects of the invention which relate to the production, stabilisation and use of the reduced forms of the phenothiazines may therefore provide significant advances in treatments employing the phenothiazines, e.g. in the treatment of conditions, diseases or disorders which are associated with oxidative damage and/or neurofilament aggregation, e.g. Alzheimer's disease, motor neurone disease, Lewy Body disease, Pick's disease and Progressive Supranuclear Palsy.

Methaemoglobinaemia is conventionally treated with methylene blue. However, in certain patients, this is ineffective. It is now apparent that this is because the methylene blue, in order to be effective, has first to be reduced to the leuco form in vivo, and that it is this leucomethylene blue that is the active species. Where a patient lacks the enzyme necessary to effect this reduction in vivo, the administration of methylene blue will be ineffective, and may even be dangerous, as large doses of methylene blue have been shown to be toxic. Thus, patients lacking the requisite enzyme can only be treated by blood transfusions.

Until now, direct administration of the reduced (leuco) form of methylene blue was impossible, as the production and stabilisation of the reduced form had not been achieved. The present invention in providing the means to produce and maintain the reduced form, enables such administration to occur. This will have benefits in ameliorating or preventing the toxicity of the oxidised form and in an ability to treat patients irrespective of deficiencies in NADPH production, e g. patients with abnormalities in the pentose phosphate pathway.

Treatment of methaemoglobinaemia with a reduced form of a phenothiazine (e.g. with a reduced form of methylene blue) has therefore been made possible by the present invention.

The present invention thus provides a method of treating methaemoglobinaemia, the method comprising the administration of a reduced form of a phenothiazine, as defined and exemplified above. The invention also provides for the use of a reduced form of a phenothiazine for the manufacture of a medicament for treating methaemoglobinaemia.

A further medical application of the reduced form of phenothiazines is the protection of tissues from oxidative damage.

Tissue damage associated with ischaemia and repercussion injury results in Fe(V)O and Fe(V)0 states of haem proteins. These proteins then facilitate the production of cytotoxic oxygen radicals whose activity leads to oxidative damage.

Studies have shown that an amelioration or prevention of such oxidative damage can be effected by the administration of riboflavin. NADPH-dependent methaemoglobin reductase catalyses the intracellular reduction of riboflavin to dihydroriboflavin (Hultquist, D. E. et al (1993) Am. J. Hematol: Jan 1993; 42(1), p. 13 et seq.). Dihydroriboflavin in turn reduces the Fe(IV)O and Fe(V)0 states of haem proteins, to prevent the formation of the radicals. Amelioration or prevention of oxidative damage associated with e.g. myocardial infarction, acute lung injury and stroke is possible.

Reduced phenothiazines such as leuco methylene blue present an alternative route to the reduction of the Fe(IV)0 and Fe(V)0 states of haem proteins. This route has only been made possible by the present invention providing the means to produce and stabilise the reduced form of these compounds.

The use of the phenothiazines in their reduced form has benefits in avoiding a dependence on NADPH and in reducing or preventing any toxicity associated with the oxidised compounds. The latter enables larger quantities of the compound to be administered.

Another instance in which oxidative tissue damage occurs is Parkinson's disease. Cohen, G. suggests that the selective and progressive destruction of the nigrostriatal dopaminergic neurones causes a compensatory increase in the dopamine turnover in the remaining dopaminergic neurones, which increase in dopamine turnover leads to an increased generation of hydrogen peroxide by monoamine oxidase (MAO). [Cohen, G. (1988) In Oxygen Radicals and Tissue Injury (ed. Halliwell, B.) pp. 130-135, FASEB, Bethesda, Maryland]. Consistent with the increased generation of hydrogen peroxide, decreased levels of reduced glutathione have been reported in the Parkinsonian brain. This results from the cellular destruction of hydrogen peroxide by glutathione reductase [Riederer, P. et al (1989) J Neurocheni52, 515-520]. High levels of hydrogen peroxide can also be generated by the auto-oxidation of dopamine [Olanow, C. W. (1990) Neurology 40, 32-371].

The excess amounts of hydrogen peroxide in the Parkinsonian brain causes oxidative damage. The hydrogen peroxide reacts with ferrous iron to form the highly cytotoxic hydroxyl radical (OH•) and the superoxide radical (O₂) which in turn causes neuronal destruction.

The present inventors contend that the leuco forms of phenothiazines can reduce the excess hydroxyl radicals or superoxide radicals in the Parkinsonian brain to decrease the amount of neurotoxic reactive oxygen species formed, thereby to protect the dopaminergic neurones from the oxidative damage and neuronal death which contributes to the disease pathology.

In various further aspects, the present invention thus provides a method of ameliorating or preventing oxidative tissue damage, and a method of treating a disease, disorder or condition selected from the group conristing of ischaemia, myocardial infarction, acute lung injury, stroke and Parkinson's disease. In each case, the methods comprise the administration of a reduced form of a phenothiazine.

The invention also provides for the use of a reduced form of a phenothiazine for the manufacture of a medicament for ameliorating or preventing oxidative tissue damage, and the use of a reduced form of a phenothiazine for the manufacture of a medicament for treating a disease, disorder or condition selected from the group consisting of ischaemia, myocardial infarction, acute lung injury, stroke and Parkinson's disease.

Various further aspects and embodiments of the present invention will be apparent to those skilled in the art in view of the present disclosure. Certain aspects and embodiments of the invention will now be illustrated by way of example only.

### Materials

Methylene Blue, thionine, glutathione, ascorbic acid, L-cysteine hydrochloride, N-acetyl L-cysteine, methionine, sodium hydrosulphite and sodium metabisulphite were all obtained from Sigma Aldrich.

### Methods and Results

### METHYLENE BLUE

### 1. Qualitative Assessment of Reduction of Methylene Blue in Aqueous Solution

In aqueous solution, the oxidised form of methylene blue has a deep blue coloration, and the reduced (leuco) form is colourless and clear. Reduction of methylene blue can therefore be determined by observation of a colour change from blue to colourless. In the present experiments, the colour change was measured by eye against a white background, but instrumentation, e.g. a visible range spectrophotometer, could also be used.

1 .1 Effect of Reducing Agents at Neutral pH

Individual reducing agents or combinations of reducing agents were added to a solution of methylene blue. Preferred reducing agents or combinations were determined. Reduction of methylene blue was assessed by visual observation of colour change. The results are shown below. The pH of the final solution of methylene blue and reducing agent(s) was about pH 6.5 in each case (here referred to as “neutral”).

| Reducing agent | Rate/extent of reduction |
|---|---|
| ascorbic acid | reduced slowly over 10-15 minutes |
| glutathione | no reduction over 15 minutes |
| sodium hydrosulphite | very rapid reduction |
| sodium metabisulphite | no reduction |
| methionine | no reduction |
| L-cysteine | no reduction |
| N-acetyl-L-cysteine | partial reduction |
| ascorbic acid solution + glutathione | rapid reduction within 5 minutes |
| solution | |

### 2 Effect at Acid or Alkaline H

Individual reducing agents or combinations of reducing agents were added, in solution; to a methylene blue solution which had been made either acid or alkaline using acetic acid or sodium hydroxide, respectively. The effect of pH on reduction of methylene blue was determined. Reduction of methylene blue was assessed by visual observation of colour change. The rate of reduction (if any) was compared to the rates of reduction at neutral pH.

In the present context, “acid” refers to a pH adjusted to approximately 3.7 using acetic acid, and “alkali” refers to a pH adjusted to approximately 9.8 using sodium hydroxide. The results are as follows:

| Reducing agent | pH | Rate of reduction |
|---|---|---|
| ascorbic acid | acid | no change in rate compared to neutral |
| ascorbic acid | alkali | no change in rate compared to neutral |
| glutathione | acid | no observable reduction (as with neutral) |
| glutathione | alkali | slight reduction over 10-15 minutes |
| ascorbic acid + | acid | fully reduces (at similar rate to neutral) |
| glutathione | | |
| ascorbic acid + | alkali | fully reduces (faster than acid and neutral) |
| glutathione | | |

1 .3 Overnight Stability of Preparations from Experiments 1 .1 and 1 .2

The stability against re-oxidation of the reduced form of methylene blue within various preparations was determined by assessing the amount of re-oxidation after overnight (16 hours) standing in air. Re-oxidation of methylene blue was determined by visual observation of colour change.

| | | Stability of reduced form to |
|---|---|---|
| Preparation | pH | overnight standing in air |
| methylene blue + ascorbic acid | neutral | very unstable |
| methylene blue + glutathione | neutral | Unstable |
| methylene blue + ascorbic acid + | neutral | Stable |
| glutathione | | |
| methylene blue + sodium | neutral | very unstable |
| hydrosulphite | | |
| methylene blue + ascorbic acid | acid | Unstable |
| methylene blue + ascorbic acid | alkali | Unstable |

### 2. Qualitative Assessment of Reduction of Methylene Blue in Aqueous Solution Using Ascorbic Acid in Combination with: Glutathione, Sodium Metabisulphite, Methionine: L-cysteine: or N-acetyl L-cysteine

2.1 Extent of reduction

The following combinations of reducing agents were added to an aqueous solution of methylene blue of 50 mg/30 ml concentration. Ascorbic acid was first added to the methylene blue. Then the glutathione, sodium metabisulphite, L-cysteine or N-acetyl L-cysteine was added. The order of addition is indicated by the relative order of the ascorbic acid and the secondary reducing agent in the following table of results. Solutions of 250 mg/3 ml reducing agent were used. Reduction of methylene blue was determined by visual observation of colour change.

| Reducing agents | Extent of reduction |
|---|---|
| ascorbic acid + glutathione | fully reduced |
| ascorbic acid + sodium metabisulphite | not quite fully reduced |
| ascorbic acid + methionine | fully reduced (slowly) |
| ascorbic acid + L-cysteine | fully reduced (quickly) |
| ascorbic acid + N-acetyl L-cysteine | fully reduced (quickly) |

2.2 Order of Addition of Ascorbic Acid and Secondary Reducing Agent

The following preparations were prepared as in Experiment 2.1. The ascorbic acid was added after the secondary reducing agent had been admixed with the methylene blue. Reduction of methylene blue was determined by visual observation of colour change.

| Reducing agents | Extent/speed of reduction |
|---|---|
| N-acetyl L-cysteine + ascorbic acid | fully reduced |
| L-cysteine + ascorbic acid | fully reduced |

2.3 Stability of Reduced form of Methylene Blue in Preparations Containing Ascorbic Acid in Combination with a Secondary Reducing Agent

The stability against re-oxidation of the reduced form of methylene blue within the preparations of Experiments 2.1 and 2.2 was determined by visually assessing the colour change from colourless to blue after 24 hours and 48 hours standing in air. The order of the ascorbic acid and secondary reducing agent in the “Preparation” column indicates the order of addition of these components to the methylene blue.

| | Stability | |
|---|---|---|
| | of reduced form | |
| Preparation | After 24 h | After 48 h |
| methylene blue + ascorbic acid + glutathione | Stable | slightly |
| | | unstable |
| methylene blue + ascorbic acid + sodium | Unstable | very |
| metabisulphite | | unstable |
| methylene blue + ascorbic acid + methionine | slightly | slightly |
| | unstable | unstable |
| methylene blue + ascorbic acid + L-cysteine | Stable | stable |
| methylene blue + ascorbic acid + N-acetyl L- | Stable | stable |
| cysteine | | |
| methylene blue + L-cysteine + ascorbic acid | Stable | stable |
| methylene blue + N-acetyl L-cysteine + | Stable | stable |
| ascorbic acid | | |

### 3 Range Finding Study for the Reduction of Methylene Blue in Solution Using Solutions of Ascorbic Acid. Glutathione. L-cysteine and N-acetyl L-cysteine

3.1 Extent of Reduction of Methylene Blue

The following studies used 1 ml of 1.67 mg/ml (50 mg/3 ml) methylene blue solution. All reducing agents were used in solutions of 83.3 mg/mi (250 mg/3 ml). Reduction of methylene blue was determined by visual observation of colour change from blue to colourless.

| | Extent/speed of reduction |
|---|---|
| Preparation | of methylene blue |
| 2:1 volume ratio of reducing agents | |
| 0.5 ml ascorbic acid + 0.25 ml L-cysteine | almost completely reduced at |
| | 10 and 15 minutes |
| 0.5 ml ascorbic acid + 0.25 ml N-acetyl L- | slightly less reduced than |
| cysteine | L-cysteine |
| 0.5 ml ascorbic acid + 0.25 ml glutathione | slightly less reduced than N- |
| | acetyl L-cysteine |
| 0.6 ml ascorbic acid + 0.3 ml L-cysteine | almost completely reduced at |
| | 10 and 15 minutes |
| 3:1 volume ratio of reducing agents | |
| 1.5 ml ascorbic acid + 0.5 ml L-cysteine | completely reduced in about 4 |
| | minutes |
| 4:1 volume ratio of reducing agents | |
| 1.0 ml ascorbic acid + 0.25 ml L-cysteine | completely reduced in about |
| | 10 minutes |
| 5:1 volume ratio of reducing agents | |
| 1.0 ml ascorbic acid + 0.20 ml L-cysteine | completely reduced in about |
| | 10 minutes |
| 1:2 volume ratio of reducing agents | |
| 0.3 ml ascorbic acid + 0.6 ml L-cysteine | not quite reduced in 10 |
| | minutes, Completely reduced |
| | by 15 minutes |

3.2 Stability of Methylene Blue in Preparations of Example 3.1

The stability against re-oxidation of the reduced form of methylene blue within the preparations of Experiment 3.1 was determined by assessing the amount of re-oxidation after the preparations had been left standing in air for 24 hours. Re-oxidation of methylene blue was determined by visual observation of colour change from colourless to blue.

| | Stability of reduced |
|---|---|
| | form of methylene blue to |
| Preparation | standing in air for 24 hours |
| 0.5 ml ascorbic acid + 0.25 ml cysteine | almost completely stable |
| 0.5 ml ascorbic acid + 0.25 ml N-acetyl L- | slightly unstable |
| cysteine | |
| 0.5 ml ascorbic acid + 0.25 ml glutathione | not fully stable |
| 0.6 ml ascorbic acid + 0.3 ml L-cysteine | Stable |
| 1.5 ml ascorbic acid + 0.5 ml L-cysteine | Stable |
| 1.0 ml ascorbic acid + 0.25 ml L-cysteine | Stable |
| 1.0 ml ascorbic acid + 0.20 ml L-cysteine | slightly unstable |
| 0.3 ml ascorbic acid + 0.6 ml L-cysteine | slightly unstable |

### 4. Long Term Stability of Methylene Blue with Ascorbic Acid+N-acetyI L-cysteine. or ascorbic acid+glutathione

The stability against re-oxidation of the reduced form of methylene blue in various preparations was determined by assessing the amount of re-oxidation after standing in air for 10 weeks, and after standing in air for 10 months. Re-oxidation of methylene blue was determined by visual observation of colour change from colourless to blue.

The volume ratio of ascorbic acid to secondary reducing agent was the same as that used in Experiment 3.2.

| | Stability after 10 weeks standing |
|---|---|
| Preparation | in air |
| ascorbic acid + N-acetyl L-cysteine | stable relative to condition after |
| | 24 hours |
| ascorbic acid + glutathione | stable relative to condition after |
| | 24 hours |
| | Stability after 10 months standing |
| Preparation | in air |
| Ascorbic acid + N-acetyl L-cysteine | stable relative to condition after |
| | 10 weeks |
| ascorbic acid + glutathione | stable relative to condition after |
| | 24 hours |

### THIONINE

### 5. Qualitative Assessment of Reduction of Thionine in Aqueous Solution

5.1 Extent of Reduction

The following reducing agents or combinations of reducing agents (250 mg/3 ml) were added to an aqueous solution of thionine (50 mg/30 mU. Reduction of thionine was determined by visual observation of colour change from blue/purple to colourless.

| Reducing agent | Rate of reduction |
|---|---|
| ascorbic acid | reduced in about 10-15 minutes |
| glutathione | no reduction |
| sodium hydrosulphite | rapid reduction |
| ascorbic acid + glutathione | rapid reduction |
| sodium hydrosulphite + glutathione | rapid reduction |

5.2 Stability of Thionine in Preparations of Experiment 5.1

The stability against reoxidation of the reduced form of thionine in the above preparations was determined by assessing the amount of re-oxidation after overnight (16 hours) standing in air. Re-oxidation of thionine was determined by visual observation of colour change.

| | Stability of reduced |
|---|---|
| | form in preparation to |
| Preparation | overnight standing in air |
| thionine + ascorbic acid | slightly unstable |
| thionine + glutathione | Unstable |
| thionine + ascorbic acid + glutathione | Stable |
| thionine + sodium hydrosulphite | Unstable |
| thionine + sodium hydrosulphite + | Unstable |
| glutathione | |

5.3 Range Finding Study for the Reduction of Thionine with Ascorbic acid and L-cysteine

All experiments used 1.67 mg/mI (50 mg/30 ml) thionine solution. Ascorbic acid, L-cysteine or glutathione was used in solution at 83.3 mg/mi (250 mg/3 ml).

| | Extent/speed of |
|---|---|
| Preparation | reduction of thionine |
| 1 ml thionine + 1 ml ascorbic acid | reduces slowly but fully |
| | (over 15 minutes) |
| 1 ml thionine + 1 ml ascorbic acid + 0.2 ml | reduces rapidly |
| L-cysteine | |

5.4 24 Hour Stability of Preparations from Example 5.3

The stability against re-oxidation of the reduced form of thionine in the preparations of Experiment 5.3 w as determined by assessing the amount of re-oxidation after standing in air for 24 hours. Re-oxidation of thionine was determined by visual observation of colour change.

| | Stability of reduced |
|---|---|
| | form of thionine to |
| Preparation | 24 h in air |
| 1 ml thionine + 1 ml ascorbic acid | Stable |
| 1 ml thionine + 1 ml ascorbic acid + 0.2 ml | Stable |
| L-cysteine | |

5.5. Long Term Stability of Thionine with Ascorbic acid+Glutathione

The stability against re-oxidation of the reduced form of thionine in a preparation containing ascorbic acid+glutathione was determined by assessing the amount of re-oxidation after standing in air for 10 weeks, and after standing in air for 10 months. Re-oxidation of thionine was determined by visual observation of colour change.

| | Stability of reduced form after 10 weeks |
|---|---|
| Preparation | standing in air |
| ascorbic acid + glutathione | Stable |
| ascorbic acid + glutathione | Stable |

1. A method of reducing an oxidised form of a pharmaceutical compound, by admixing the oxidised form of the compound with ascorbic acid and with at least one sulphydryl compound.

2. A method of stabilising the reduced form of a pharmaceutical compound which can exist in both oxidised and reduced forms by mixing said pharmaceutical compound with ascorbic acid and with at least one sulphydryl compound.

3. A method according to claim 1 or claim 2, in which the sulphydryl compound is an sulphur-containing amino acid or a peptide including at least one amino acid unit derived from such an amino acid, or a derivative of such an amino acid or peptide.

4. A method according to claim 3, in which said derivative is a salt, ester or amide.

5. A method according to claim 3 or 4, in which said amino acid is cysteine or methionine.

6. A method according to claim 1, in which the sulphydryl compound is glutathione, cysteine, N-acetyl-cysteine, methionine, or a mixture of any two or more thereof.

7. A method according to any one of the preceding claims, in which the pharmaceutical compound is a phenothiazine.

8. A method according to claim 7, in which the phenothiazine is Toluidine Blue C, Thionine, Azure A, Azure B, Azure C, Methylene Blue or 1,9-Dimethyl-methylene Blue, or a mixture of any two or more thereof.

9. A method according to any one of claims 1 to 6, in which the pharmaceutical compound is riboflavin, a ubiquinone, 4,7-phenanthroline-5, 6-hydroquinone or dapsone.

10. A method according to any one of the preceding claims, in which the weight ratio of ascorbic acid to the pharmaceutical compound is from about 10:1 to about 100:1.

11. A method according to any one of the preceding claims, in which the weight ratio of sulphydrl compound(s) to the pharmaceutical compound is from about 2:1 to about 200:1.

12. A method according to any one of the preceding claims, in which the weight ratio of sulphydryl compound to ascorbic acid is from about 1:0.5 to about 1:5.

13. The use of a reduced form of a phenothiazine, stabilised by a method according to any one of the preceding claims, for the manufacture of a medicament for treating methaemoglobinaemia.
